(19) United States
(12) Patent Application Publication
(10) Pub. No.: US 2006/0173246 A1
(43) Pub. Date: Aug. 3, 2006

(54) Medical information interface and communication system

(76) Inventor: John R. Zaleski, West Brandywine, PA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/346,488

(22) Filed: Feb. 2, 2006

Related U.S. Application Data
(60) Provisional application No. 60/649,224, filed on Feb. 2, 2005.

(51) Int. Cl. A61B 5/00

(52) U.S. Cl. 600/300; 128/920

(57) ABSTRACT

A mobile point-of-care medical station adapted to be employed by patient monitors made by a variety of different manufacturers. A mobile point-of-care medical station includes a patient medical parameter processor and a communication interface. The patient medical parameter processor automatically acquires data, representing medical parameters of a patient, and processes the patient medical parameter data for presentation to a user on a display. The communication interface enables communication with remote systems via a network and communicates patient medical parameter data to a remote system by formatting patient medical parameter data into multiple individual messages. An individual message incorporates a patient identifier and a communication address. The communication address is associated with a particular communication interface of a particular mobile point-of-care medical station, and enables the remote system to identify the particular mobile point-of-care medical station from multiple different mobile point-of-care medical stations.

### CROSS-REFERENCE TO RELATED APPLICATIONS

The present application is a non-provisional application of provisional application having Ser. No. 60/649,224 filed by John R. Zaleski on Feb. 2, 2005.

### FIELD OF THE INVENTION

The present invention generally relates to computer information systems. More particularly, the present invention relates to a medical information interface and communication system.

### BACKGROUND OF THE INVENTION

Computer information systems (“systems”) include computers that communicate with each other over a network, such as the Internet, and computers that manage information. For example, a healthcare enterprise uses systems to store and manage medical information, such as data, representing medical information, for patients in their care. Examples of the medical information include a patient's vital signs, such as pulse, blood pressure, temperature, respiratory rate, and oxygen saturation. Prior systems for monitoring the medical information included manual and automatic monitoring methods.

In the prior manual system, a person, such as a nurse, manually measures or reads a patient's vital signs and manually records the related data in a patient's paper medical record (i.e., chart). Disadvantages of the prior manual system include, for example, being slow, being time consuming, being expensive, being error prone, providing limited access, and providing limited trend analysis. The prior manual system is slow, time consuming, and expensive because it is performed by human labor. The prior manual system is prone to error because a person may mistakenly record incorrect data for the patient. The prior manual system permits a limited number of clinicians to access the paper record because the paper record is typically located at a nurse station closest to the patient's bed or outside the patient's room. The prior manual system limits historical trend analysis of the recorded data because it is difficult and time consuming for a person to manually analyze the recorded data in multiple ways.

In the prior automatic system, a proprietary software interface manually measures or reads a patient's vital signs and automatically records the related data in a patient's digital medical record. Although the prior automatic system improves upon several disadvantages of the prior manual system, disadvantages of the prior automatic system include being expensive and complex. The prior automatic system is expensive and complex because they use expensive interface servers to connect patient monitors with healthcare information systems. The expensive interface servers use complex software programming and proprietary communication interface modules (including software and hardware). The proprietary communication interface modules are typically limited to a specific monitor manufacturer, thereby reducing or eliminating the economies of scale and commonality provided by present non-proprietary communication interfaces.

Accordingly, there is a need for a medical information interface and communication system that overcomes these deficiencies and related problems of the prior systems.

### SUMMARY OF THE INVENTION

A mobile point-of-care medical station for automatically recording a patient's medical information, without a proprietary hardware and software communication interface. A mobile point-of-care medical station includes a patient medical parameter processor and a communication interface. The patient medical parameter processor automatically acquires data, representing medical parameters of a patient, and processes the patient medical parameter data for presentation to a user on a display. The communication interface enables communication with remote systems via a network and communicates patient medical parameter data to a remote system by formatting patient medical parameter data into multiple individual messages. An individual message incorporates a patient identifier and a communication address. The communication address is associated with a particular communication interface of a particular mobile point-of-care medical station, and enables the remote system to identify the particular mobile point-of-care medical station from multiple different mobile point-of-care medical stations.

### BRIEF DESCRIPTION OF THE DRAWINGS

FIG. 1 illustrates a medical information communication system (“system”), in accordance with invention principles.

FIG. 2 illustrates a host computer for use with the system, as shown in FIG. 1, in accordance with invention principles.

FIG. 3 illustrates a user interface image window providing data initiation for the host computer, as shown in FIG. 2, in accordance with invention principles.

FIG. 4 illustrates a method of communication for use with the system, as shown in FIG. 1, in accordance with invention principles.

FIG. 5 illustrates a boot agent for the host computer's application, in accordance with invention principles.

FIG. 6 illustrates user interface image window providing data results for the host computer, as shown in FIG. 2, in accordance with invention principles.

### DETAILED DESCRIPTION OF THE PREFERRED EMBODIMENTS

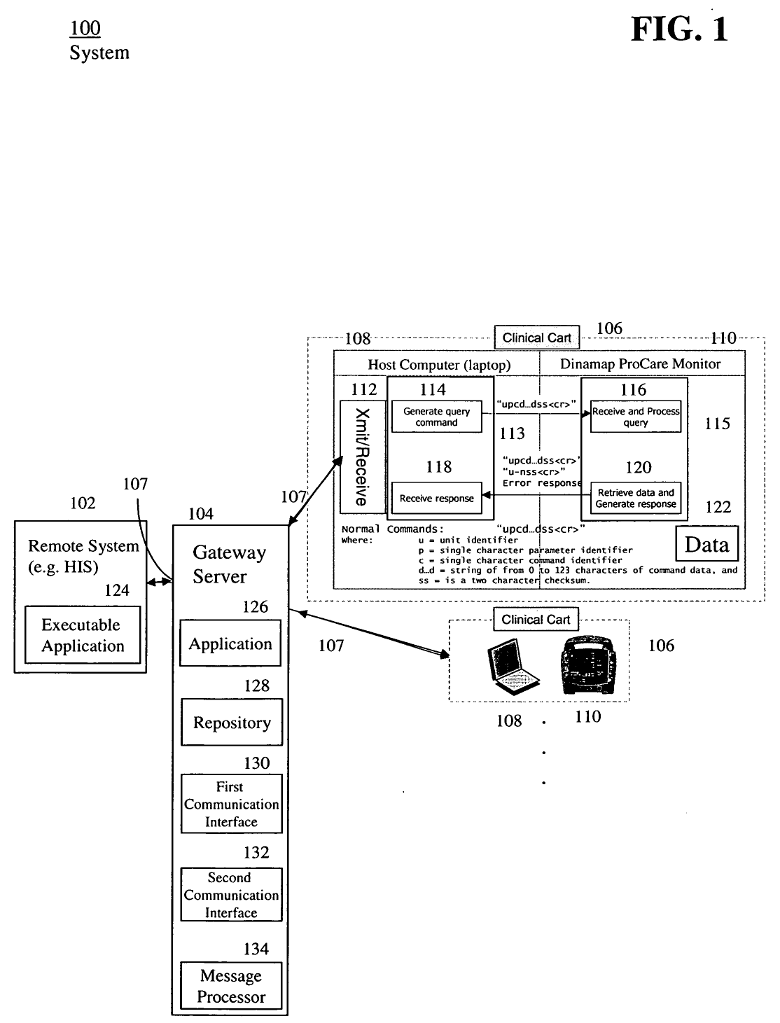

FIG. 1 illustrates a medical information communication system (“system”) 100. The system 100 includes a remote system 102, a gateway server 104, and one or more clinical carts 106. A communication path 107 interconnects elements of the system 100.

The system 100 may be employed by any type of enterprise, organization, or department, such as, for example, providers of healthcare products and/or services responsible for servicing the health and/or welfare of people in its care. For example, the system 100 includes a healthcare information system (“HIS”). A healthcare provider provides services directed to the mental, emotional, or physical well being of a patient. Examples of healthcare providers include a hospital, a nursing home, an assisted living care arrangement, a home health care arrangement, a hospice arrangement, a critical care arrangement, a health care clinic, a physical therapy clinic, a chiropractic clinic, a medical supplier, a pharmacy, a doctor's office, and a dental office. When servicing a person in its care, a healthcare provider diagnoses a condition or disease, and recommends a course of treatment to cure the condition, if such treatment exists, or provides preventative healthcare services. Examples of the people being serviced by a healthcare provider include a patient, a resident, a client, and an individual.

The system 100 and/or elements contained therein also may be implemented in a centralized or decentralized configuration. The system 100 may be implemented as a client-server, web-based, or stand-alone configuration.

The system 100 elements and/or processes contained therein may be implemented in hardware, software, or a combination of both, and may include one or more processors, such as processor 104. A processor is a device and/or set of machine-readable instructions for performing task. The processor includes any combination of hardware, firmware, and/or software. The processor acts upon stored and/or received information by computing, manipulating, analyzing, modifying, converting, or transmitting information for use by an executable application or procedure or an information device, and/or by routing the information to an output device. For example, the processor may use or include the capabilities of a controller or microprocessor.

The communication path 107 (otherwise called network, bus, link, connection, channel, etc.) represents any type of protocol or data format. The protocol or data format includes, but is not limited to, one or more of the following: an Internet Protocol (IP), a Transmission Control Protocol Internet protocol (TCPIP), a Hyper Text Transmission Protocol (HTTP), an RS232 protocol, an Ethernet protocol, a Medical Interface Bus (MIB) compatible protocol, a Local Area Network (LAN) protocol, a Wide Area Network (WAN) protocol, a Campus Area Network (CAN) protocol, a Metropolitan Area Network (MAN) protocol, a Home Area Network (HAN) protocol, an Institute Of Electrical And Electronic Engineers (IEEE) bus compatible protocol, a Digital and Imaging Communications (DICOM) protocol, and a Health Level Seven (HL7) protocol.

The remote system 102 includes an executable application 124 executing on a server. In the case of the client-server or web-based configurations, the executable application 124 may be accessed remotely over a communication network, represented by communication path 107.

The gateway server 104 (e.g., given the proprietary term “Dinacom”) includes a gateway executable application 126, a repository 128, a first communication interface 130, a second communication interface 132, and a message processor 134. The gateway server 104 represents an interface between the remote system 102 and one or more clinical carts 106. The interface includes hardware (e.g., a gateway server) and/or software (e.g., a gateway application).

The repository 128 stores data associating multiple mobile point-of-care medical station 106 identifiers with corresponding multiple communication interface communication addresses.

The first communication interface 130 enables communication with multiple mobile point-of-care medical stations 106 via the network 107. The first communication interface 130 receives patient medical parameter data from an individual medical station 106 formatted as an individual message incorporating a patient identifier and a communication address. The formatted individual message is compatible with a Health Level 7 (HL7) transaction data format. The communication address is associated with a particular communication interface of a particular mobile point-of-care medical station 106.

The first communication interface 130 receives patient medical parameter data and associated local workstation identification information to uniquely establish a link between patient, medical monitor 110, and raw parameters ensuring no loss of patient specificity.

The second communication interface 132 enables communication of received data to a remote system 102, such as a medical information system.

The message processor 134 uses the repository 128 for determining a particular mobile point-of-care medical station 106 associated with a particular received individual message.

The first 130 and second 132 communication interfaces employ first and second separate operational processes respectively. The first process manages inbound communications from mobile point-of-care medical stations 106. The second process manages outbound communications to the remote system 102. The separate first and second processes advantageously prevent data queuing interaction between inbound and outbound communications. In one example, the separate first and second processes are separate threads of operation.

The clinical cart 106 represents a point-of-care medical station (“medical station”) including a host computer 108 and a patient monitor 110. The clinical cart 106 may be fixed or mobile. When the clinical cart 106 is mobile, it represents a mobile point-of-care medical station (“mobile station”).

The host computer 108 further includes a communication interface 112 (e.g., transmit/receive) and an acquisition processor 113, which includes a query generator 114 and a response receiver 118.

The host computer 108 may be fixed and/or mobile (i.e., portable). The host computer 108 may be implemented in a variety of forms including, but not limited to, one or more of the following: a personal computer (PC), a desktop computer, a laptop computer, a workstation, a minicomputer, a mainframe, a supercomputer, a network-based device, a personal digital assistant (PDA), a smart card, a cellular telephone, a pager, and a wristwatch.

The patient monitor 110 further includes a query processor 115 and data 122, representing the patient's medical information. The query processor 115 further includes a query receiver 116 and a response generator 120.

The patient monitor 110 represents a source and of any data 122 or other information that may be needed or used by the system 100. The data 122 may be pushed to the system 100 and/or pulled by the system 100, automatically and/or manually, at one time, periodically, or as needed. The data 122 may have any format and may represent any type of information. For example, the data 122 represents any type of medical information for a patient, such as, for example, patient vitals, blood pressure, pulse, respiration rate, and oxygen saturation.

The host computer 108 is physically electrically coupled to the patient monitor 110 by any method including, for example, a wired (e.g., serial or parallel cable, such as a RS-232 cable) or wireless connection (e.g., radio or infra red frequency) that permits communication to occur between the host computer 108 and the patient monitor 110.

During communication between the host computer 108 and the patient monitor 110, the query generator 114 in the host computer 108 generates and sends a query command to the patient monitor 110. The query command represents a request for data 122, representing the medical information, by the host computer 108 from the patient monitor 110. The query receiver 116 in the patient monitor 110 receives and processes the query command. The response generator 120 in the patient monitor 110 retrieves the requested data 122 from a memory device located in the patient monitor 110, and generates and sends a response to the host computer 108. The response receiver 118 in the host computer 108 receives and processes the response. After the host computer 108 has retrieved the requested data 122 from the patient monitor 110, the host computer 108 may communicate the requested data 122 to the remote system 102 via the communication interface 112 (e.g., wired or wireless) in the host computer 108 and via the gateway 104.

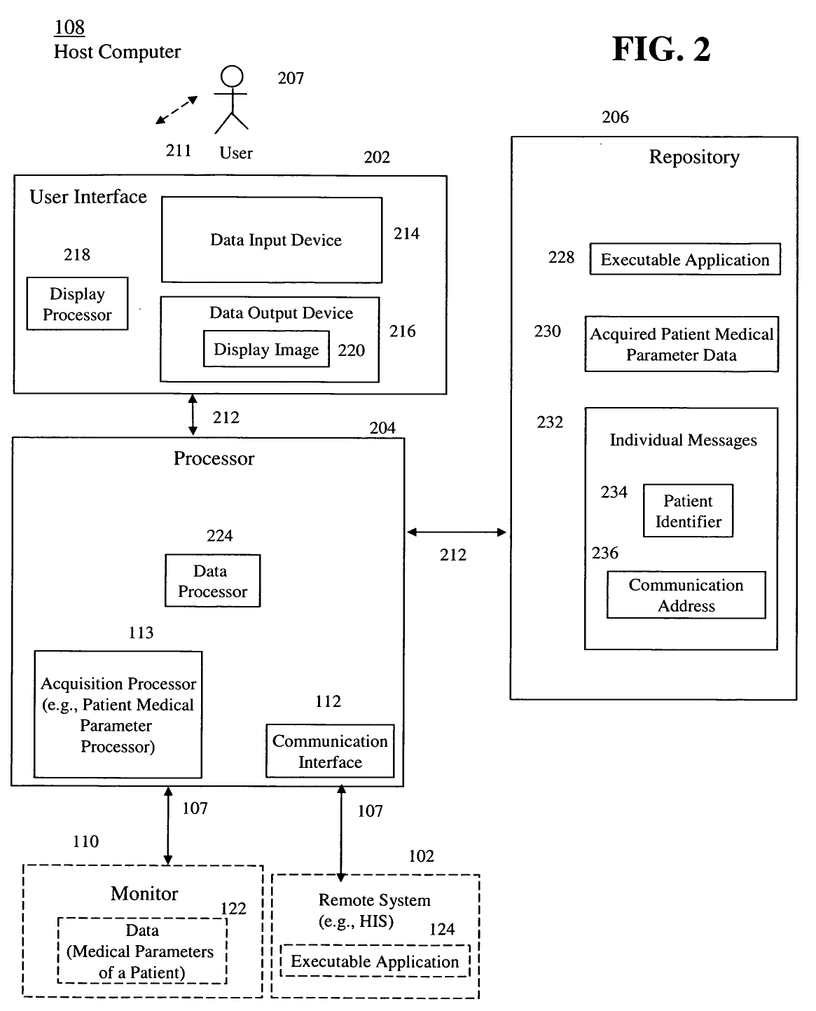

An executable application 228 (FIG. 2), otherwise called a system interface application, retrieves the data 122 from the patient monitor 110. In one example, the executable application 228 resides locally on the host computer 108, and in another example, it resides elsewhere in the network 107.

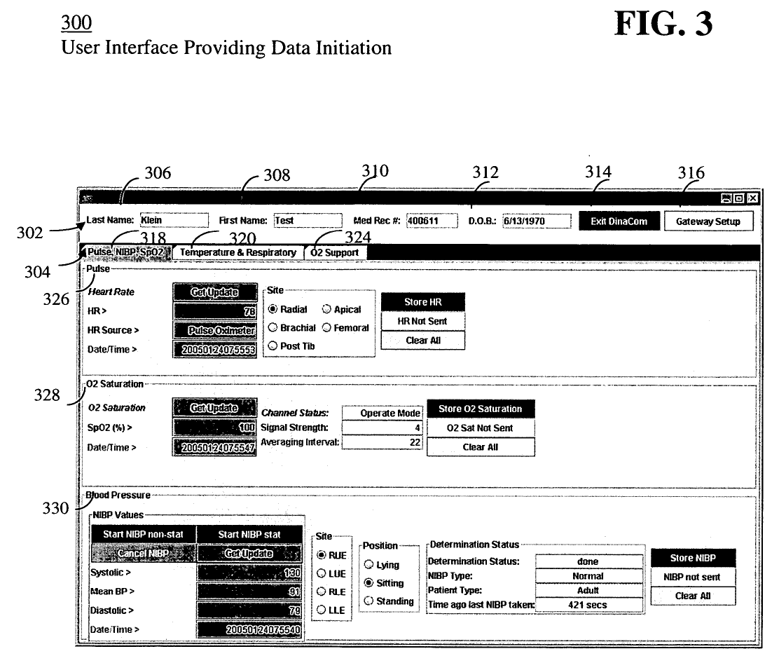

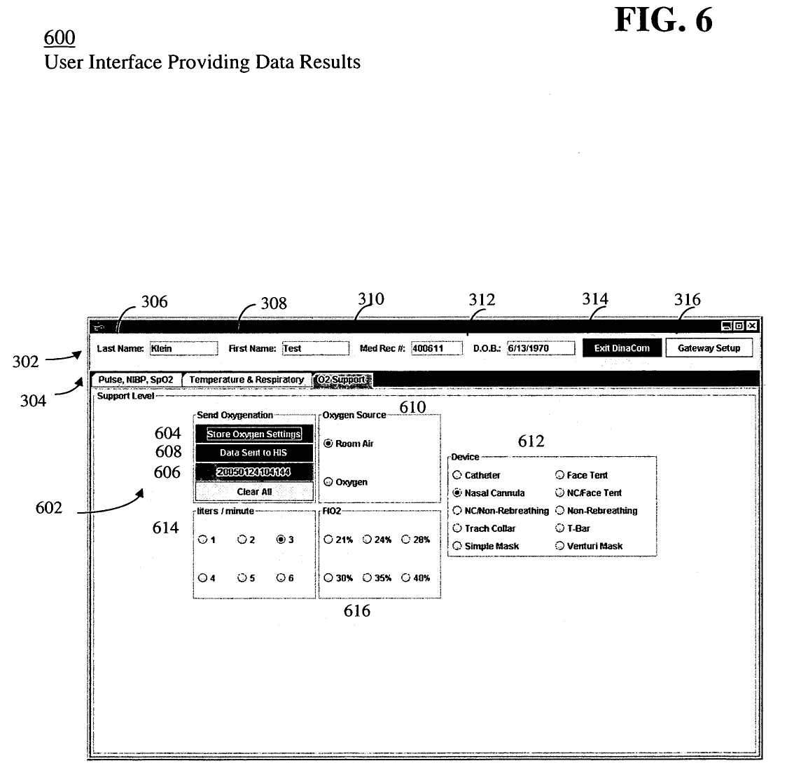

The executable application 228 is launched, for example, using a Web-based boot agent that is activated by a button click from within a Net Access application, for example. Once inside the Net Access application, a patient record is selected. Within the selected patient record, a hyperlink (e.g., universal resource locator (URL)) to a boot agent for the executable application 228 provides a link from the Net Access application to the boot agent residing on the host computer 108. This launches the executable application 228. Parameters are carried with the URL call to the executable application 228, enabling patient name, medical record number, and date of birth to be displayed locally within a display window (see FIGS. 3 and 6), associated with the executable application 228.

The host computer 108 communicates over standard Ethernet (e.g., wireless or wired) to a gateway application, which resides on a gateway server 104 that is not necessarily co-located with the clinical carts 106. A many-to-one relationship exists between the clinical carts 106 and the gateway server 104, such that the gateway server 104 can accept multiple concurrent connections (e.g., through the use of multithreading). Transactions that are received from each clinical cart 106 are queued for delivery to the remote system 102 (e.g., an HIS interface engine).

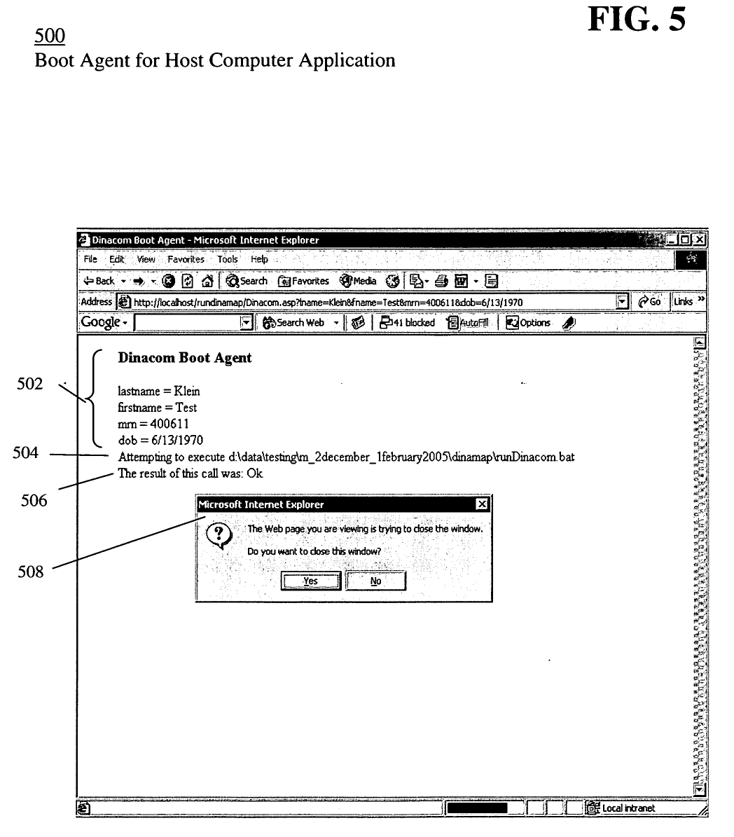

As transactions are received at the gateway application 126 from each clinical cart 106, bidirectional checksum verification is performed together with an acknowledgement transmission. The communication interface 112 in the host computer 108 sends raw patient data 122 to the gateway's application 126. The patient data 122 arrives at the gateway server 104 together with a checksum calculated by the host computer's application 228. The gateway application 126 computes the resultant checksum using the same algorithm as that contained within the host computer's application 228. The gateway application 126 compares the computed checksum with the checksum just received on that particular message. If the two checksums match, an acknowledgement message is created, and sent back to the host computer's application 228 to notify it that the message was properly received at the gateway server 104. A properly received message is reflected in the user interface on the host computer's application 228 (see 506 in FIG. 5). The gateway application 126 translates the received message into an appropriately formatted HL7 message, queues the formatted message in order of arrival (FIFO) for delivery to the remote system 102, and transmits the queued message to the remote system 102.

FIG. 2 illustrates a host computer 108 for use with the system 100, as shown in FIG. 1. The host computer 108 includes a user interface 202, a processor 204, and a repository 206. A user 207, the remote system 102, and the patient monitor 110 interact with the host computer 108.

A communication path 212 interconnects elements of the host computer 108 and communication path 107 interconnects the host computer 108 with the remote system 102 and the patient monitor 110. The communication path 212 may be the same as or different from the communication path 107. The dotted line near reference number 211 represents interaction between the user 207 and the user interface 202.

The user interface 202 further provides a data input device 214, a data output device 216, and a display processor 218. The data output device 216 further provides one or more display images 220, which are presented for viewing by the user 207.

The processor 204 further includes an acquisition processor 113, otherwise called a patient medical parameter processor, a communication interface 112, and a data processor 224.

The repository 206 further includes an executable application 228, acquired patient medical parameter data 230, and individual messages 232, which include a patient identifier 234 and a communication address 236.

The user interface 202 permits bidirectional exchange of data between the host computer 108 and the user 207 of the host computer 108 or another electronic device, such as a computer or an application, for example.

The data input device 214 typically provides data to a processor in response to receiving input data either manually from a user or automatically from another electronic device. For manual input, the data input device is a keyboard and a mouse, but also may be a touch screen, or a microphone and a voice recognition application, for example.

The data output device 216 typically provides data from a processor for use by a user or another electronic device. For output to a user, the data output device 216 is a display, such as, a computer monitor or screen, that generates one or more display images 220 in response to receiving the display signals from the display processor 218, but also may be a speaker or a printer, for example. The display images 220 provide information in any format including information used by a healthcare enterprise, such as any information/data stored in the repository 206. Examples of display images 220 include, for example, text, graphics, photos, images, graphs, charts, forms, etc.

The display processor 218 (e.g., a display generator) includes electronic circuitry or software or a combination of both for generating the display images 220 or portions thereof in response to receiving data representing display images, which may be stored in the repository 206. The data output device 216, implemented as a display, is coupled to the display processor 218 and displays the generated display images 220. The display images 220 provide, for example, a graphical user interface, permitting user interaction with the processor 204 or other device. The display processor 218 may be implemented in the user interface 202 and/or the processor 204.

The acquisition processor 113 acquires data 122. The acquisition processor 113 may acquire the data directly or through the communication interface 112. The processor 204 (e.g., data processor 224 in processor 104) stores the data 122, which was acquired, as acquired data 230 in the repository 206.

The communication interface 112 manages communications within and outside the host computer 108, such as, for example, with the patient monitor 110 and the remote system 102.

The data processor 224 performs general data processing for the host computer 108.

The repository 206 represents any type of storage device, such as a computer memory device or other tangible storage medium, for example. The repository 206 may be implemented as a database, for example. The repository 206 represents one or more memory devices, located at one or more locations, and implemented as one or more technologies, depending on the particular implementation of the host computer 108.

An executable application, such as the executable application 228, the executable application 124 (FIG. 1) and/or the gateway application 126, comprises machine code or machine readable instruction for implementing predetermined functions including, for example, those of an operating system, a software application program, a healthcare information system, or other information processing system, for example, in response user command or input.

An executable procedure is a segment of code (i.e., machine readable instruction), sub-routine, or other distinct section of code or portion of an executable application for performing one or more particular processes, and may include performing operations on received input parameters (or in response to received input parameters) and providing resulting output parameters.

The acquired data 230 represents the data 122 acquired by the acquisition processor 113 and stored in the repository 206.

The individual messages 232 include the patient identifier 234 and the communication address 236.

FIG. 3 illustrates a user interface image window 300 providing data initiation for the host computer 108, as shown in FIG. 2. FIG. 3 shows the user interface for the host computer's 108 executable application 228, after being launched by the boot agent for the executable application 228. The user interface image window 300 includes header information 302 and medical information 304.

The header information 302 includes, for example, a patient's last name 306, the patient's first name 308, the patient's medical record number (“MRN”) 310, and the patient's date of birth (“DOB”) 312. The header information 302 includes an exit gateway (e.g., Dinacom) button 314 and a gateway set up button 316.

The medical information 304 includes, for example, the patient's vital signs, such as, for example, pulse 326, blood pressure 330, and oxygen saturation 328, temperature and respiratory 320, and oxygen support 324. The medical information 304 may be organized, such as by tabs, menus, tables, etc. for convenient selection and use. In FIG. 3, a tab 318 for pulse, blood pressure, and oxygen saturation is selected, which shows detailed information in display areas for each of the pulse 326, blood pressure 330, and oxygen saturation 328.

The user interface image window 300 advantageously permits a user to describe, select, and control data 122, representing the patient's medical information, which is to be retrieved by the host computer 108.

Data related features shown in FIG. 3 for the patient's pulse in the pulse display area 326 include, for example, a heart rate, a heart rate source, a date/time duration, a site (e.g., radial). Command functions for the patient's pulse include “Get update,” “store HR,” “HR not sent,” and “clear all.”

Data related features shown in FIG. 3 for the patient's oxygen saturation in the oxygen saturation display area 328 include, for example, oxygen saturation percentage, date/time, channel status, signal strength, and averaging interval. Command functions for the patient's oxygen saturation include “Get update,” “store oxygen saturation,” “oxygen saturation not sent,” and “clear all.”

Data related features shown in FIG. 3 for the patient's blood pressure in the blood pressure display area 330 include, for example, systolic, mean blood pressure (BP), diastolic, date/time, site (e.g., right upper extremity (RUE)), position (e.g., sitting), and determination status (e.g., determination status, non-invasive blood pressure (NIBP) type, patient type, and time since last NIBP taken). Command functions for the patient's pulse include “Get update,” “store NIBP,” “NIBP not sent,” and “clear all.”

A patient medical parameter processor 204 (FIG. 2) communicates directly with medical monitors 110 through a serial connection, for example, to retrieve patient medical parameters 122. The patient medical parameters 122 are associated with local workstation identification information to uniquely establish a corresponding link between a particular patient, a particular patient monitor 110, and particular raw parameters 122 to ensure no loss of specificity for patient.

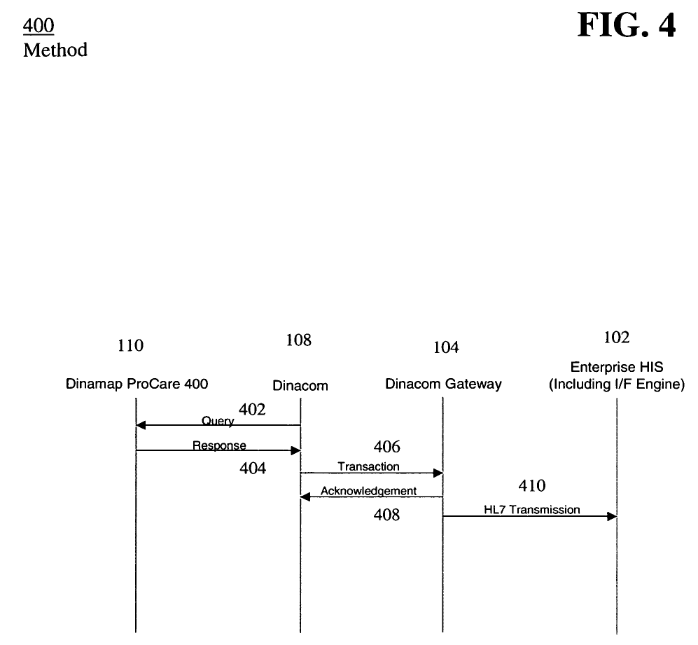

The application 228 in the host computer 108 receives elements, passed to it via the boot agent of the application's 228. These elements include [LastName], [FirstName], [MRN], and [DOB]. After the application 228 receives the elements, the interaction between the functions and the patient monitor 110 or the gateway server 104 are user driven, as specified under area 304 in FIG. 4 (i.e., workflows are specific to the type of data to be retrieved, parsed, and transmitted).

A feature of the application 228 involves communication setup between the host computer 108 in physical proximity with the patient monitor 110 and the gateway server 104. The system 100 identifies, at a single time, the identity of each host computer 108 to the gateway server 104 on the remote network 102. It is not necessary to later define the host computer's 108 identity to the gateway server 104. The single time identity is accomplished by the gateway server 104 reading a communication address included in a raw data transmission, otherwise called a data vector, sent by each application 228 on a corresponding host computer 108 to the gateway server 104.

The data vector comprises the patient-specific information 234 (i.e., patient identifier) (FIG. 2), and local network and processor identifying information 236 (e.g., a communication address) (FIG. 2). The patient-specific information 234 includes patient information that is both collected (FIG. 6) and manually entered (FIG. 3) for each patient. The communication address is used to establish a communication dialogue between the application 228 on the host computer 108 and the gateway application 126.

An example of a data vector is shown, as follows:

RESPIRATORY-PANEL∥fn=Test|ln=Klein|mr=400611|db=6/13/1970|rs=18|dt=20050123144225|_{—}9200_[MLJRZ0C/192.168.1.105]

The font size may be reduced in order to fit the entire Vector on a single line. In parsing this Vector, similar to HL7 formatting, individual elements are separated by a vertical bar, or “|”.

The first piece of information is the name of the panel 304 from which the data were sourced (RESPIRATORY-PANEL). This information is used by the gateway application 126 for further processing, as described herein. The source entry is followed by two vertical bar symbols. These provide a clear separation point between the source panel and the remaining data. The remaining data are either patient or local workstation specific.

The next piece of relevant information is the patient's first name, fn=Test. Each field has a unique two-character identifier employed to identify the data 122. This information, together with the panel name, serves to direct specific processing within the gateway application 126. The two-character identifiers are identified as they arise. Several of the two-character identifiers are defined in the following table:

| Parameter | |
|---|---|
| Code | Definition |
| fn | First name |
| ln | Last name |
| mr | Medical record number |
| db | Date of birth |
| rs | Respiratory rate |
| dt | Date & time stamp |

The last data element appended to a data vector transmitted by the application 228 in the host computer 108 to the gateway server 104 is the Internet Protocol (IP) address of the host computer 108 and the communications port. In the vector above, the address and port appear as:
_{—}9200_[ML2JRZ0C/192.168.1.105]

The gateway application 126 uses this information to communicate back to the host computer 108. By receiving this information, there is an established link between medical record number 310 and local host computer 108. The port on which the gateway application 126 communicates back to the client is 9200, and the IP address and resolved name are 192.168.1.105/ML2JRZ0C, respectively.

Each data vector contains the same class of starting and finishing components, wherein the sub-panel identifier starts the data vector and the port/IP address terminates the vector.

The coding is similar for transmissions. However, the placement, type of data, and location of the fields within the data vector can change depending on the type of data being sent (e.g., NIBP, pulse, temperature, etc.).

Therefore, by knowing the source panel for each data vector (i.e., the sub-panel identifier leading off the data vector), the gateway application 126 can route transactions more efficiently to specific functions for parsing and computing.

In one example, patient data 122 is automatically collected and displayed on a mobile cart laptop-based user interface. The patient data 122 is also sent to the gateway server 104. Individual transactions (i.e., messages) from individual mobile carts 106 contain patient identifying information 234 so no loss of specificity or hazard associated with miss-identifying patients can occur. The IP address and port of the source cart are also transmitted with the data 122 so that the gateway knows automatically from which the data originated, thereby removing the need for each cart 106 to manually identify itself on the network 107.

The gateway server 104 can connect with as many carts 106 (i.e., clients) as necessary, without causing a connection queue. This is done by running two separate threads: one inbound from each cart 106, and another outbound from each cart 106 that establishes a connection with the remote system 102.

FIG. 4 illustrates a method 400 of communication for use with the system 100, as shown in FIG. 1. The method 400 may also be described as an event trace for data communications from the executable application 228 in the host computer 108 to a clinical information repository residing in the remote system 102. The event trace generally flows from the host computer 108, to the patient monitor 110 via event 402, back to the host computer 108 via event 404, to the gateway via event 406 with an acknowledgement provided back to the host computer via event 408, and to the remote system via event 410.

At events 402 and 404, executable application 228 in the host computer 108 engages in a query-response dialogue with the patient monitor 110, as described with reference to FIG. 1, to acquire the data 122. After the executable application 228 in the host computer 108 receives the data 122, the executable application 228 updates user interface image windows 300 in FIG. 3, 500 in FIG. 5, and 600 in FIG. 6.

At event 406, the executable application 228 in the host computer 108 formats and transmits the data 122 to the gateway 104.

At event 408, the gateway responds to the receipt of the data 122 by sending back to the executable application 228 in the host computer 108 an ACK (i.e., acknowledgement) message or NACK (i.e., negative acknowledgement) message, depending on the success or failure, respectively, of the data transmission. When the gateway 104 transmits an ACK message back to the executable application 228 in the host computer 108, the gateway 104 formats an HL7 results transaction and transmits the formatted HL7 results transaction to the remote system 102.

FIG. 5 illustrates a user interface image window 500 for a boot agent executable procedure for the application 228 in the host computer 108. The user interface image window 500 includes a summary 502 of the data vector information as provided in the header 302 (FIG. 3), executable data 504, a message 506 stating whether the acknowledgement was ok, and an Internet Explorer® script box 508.

The patient-specific information 234 contained within the data vector is provided by a net application 228. In one example, a net access application is configured with a URL link to the boot agent for the net application 228. The boot agent retrieves information passed to it by the net access application through hypertext transmission protocol (HTTP) parameters. The boot agent creates a launching script with the HTTP parameters and initiates the execution of the application 228 on the host computer 108.

FIG. 5 illustrates the user interface image window 500 for the boot agent page just after launching the net application 228. A script box 508 within the boot agent page initiates closing of the page so that when the user 207 exits the application 228, the user 207 merely clicks on the “Yes” button in the script box 508. The script box 508 is a standard Internet Explorer® warning indicating that the page is being asked to be closed by someone other than the initiator, and is part of normal operation.

FIG. 6 illustrates user interface image window 600 providing data results 602 for the host computer 108, as shown in FIG. 2. The data results provide transmission of a data vector, representing a patient's vital medical parameter 122.

Storing data initiates the creation of a data vector, as described herein, with components built from the content 302 and 304 (FIG. 3) of the user interface image window 300.

In FIG. 6 (e.g., O2 support), when the user selects the “store oxygen settings” button 604, the date/time field 606 is updated (e.g., 20050124104144 for Jan. 24, 2005, 10:41:44). When an acknowledgement is received by the application 228 that the data was successfully sent to the remote system 102, a success message (e.g., “Data Sent to HIS”) 608 is displayed.

The data results 602, transmitted in this particular instance, are displayed in the following areas: Oxygen Source (e.g., “Room Air”) 610, Device (e.g., “Nasal Cannula”) 612, and Minute Volume (e.g., “3 liters/minute”) 614. No data results are displayed for Inspired O2 Fraction (i.e., FIO2) 616, in this example.

The associated data vector transmitted to the gateway application 126 is as follows: Incoming data: OXYGEN-PANEL∥fn=Test|ln=Klein|mr=400611|db=6/13/1970|dt=20050124125635|o2=ROOM AIR|lp=5|fi=|dv=NASALCAN|_{—}9200_[ML2JRZ0C/165.226.242.70]

The checksum transmitted by the host computer 108 to the gateway server 104 with the data vector is ninety, for example. The checksum computed by the gateway application 126 is also ninety, thereby confirming a complete record. An acknowledgement is sent back to the host computer 108, resulting in the success message appearing within the text field below the storage button.

The individual elements of data transmitted across (together with their two-character field codes) are as follows:

fn=[Test]

In=[Klein]

mr=[400611]

db=[6/13/1970]

dt=[20050124125635]

o2=[ROOM AIR]

lp=[5]

fi=[ ]

dv=[NASALCAN]

The HL7 transmission from the gateway application 126 to the remote system 102 are as follows: MSH|ˆ˜\&|Dinamap∥INVISION∥20050124125635∥ORUˆR01|20050124125635|P|2.3 PID|1∥400611ˆˆˆˆExternalPatientID∥KleinˆTest∥∥∥∥∥|400611 OBR|1∥ˆVitals∥|20050124125635∥∥∥∥∥∥∥∥∥∥|20050124125635 OBX|1|ST|ˆSOURCE∥ROOM AIR∥∥∥R∥∥∥|OBX|2|ST|ˆMV∥5|liters/minute∥∥|R∥∥∥|OBX|3|ST|ˆFIO2∥|%∥∥|R∥∥∥|OBX|4|ST|ˆDEVICE∥NASALCAN∥∥∥R∥∥∥|

The HL7 message is may be changed in format and style to suit the needs of an existing remote system 102.

The processing within the gateway server 104 advantageously employs two separate threads: the Connect processing thread for HL7 messages, and a Handler thread for accepting socket connections from the application 228 in the host computer 108. The design of the gateway server 104 is such that as a new host computer application 228 comes on line and attempts to connect with the gateway server 104, a new thread is spawned to “handle” the traffic from that new host computer application 228. Meanwhile, as a host computer application 228 is sending traffic to the gateway server 104, the data is funneled into a Vector queue, which is passed to the Connect processing thread that transmits an individual element within the queue, and removes elements from the processing list once completed. In this way, a single TCP/IP socket connection is maintained between the gateway server 104 and the remote system 102, while the gateway server 104 supports multiple host computer applications 228. This advantageously permits individual host computer applications 228 to have a dedicated connection and minimal wait time, while the data traffic is then processed back through the single networking connection that passes data 122 in the form of HL7 formatted results to the remote system 102.

The system may use, for example, one of the following:

1. Java virtual machine available free for download from http://www java.com;

2. Java communications library (javacomm20-win32.zip), also available free for download from http://www.java.sun.com.

The system 100 includes the following advantages over the prior systems. The system 100 eliminates the need for a proprietary hardware and software communication interface, which reduces cost and complexity, and increases efficiency. The system 100 provides integration of the user interface with patient vital sign monitors mounted on mobile carts. The system 100 uses industry standard communication protocols. The system 100 has minimal configuration and setup tasks. The system 100 eliminates transcription errors, provides information concurrently for display, and can help other workflow applications to trend patient vital sign results with other clinical information, such as fluid input/output (I/Os), nursing progress notes, and relevant tests results.

Monitors of a variety of different manufacturers may employ the system 100. Nurses may use the system's user interface image windows 300, 500, 600 to collect patient vital sign information efficiently and effectively. Patient care becomes more efficient as the vital sign data is available for display by multiple varied clinical users (doctors, nurses, other care givers) almost simultaneously with the collection event and at a variety of different locations. The system 100 connects multiple patient vital sign monitors (e.g., BP, Pulse, O2) with hospital information systems, such as a clinical information system. The system 100 collects information with streamlined actions by a user 207 to identify a patient and record observations that are sent to a remote system 102 storage and/or display.

Hence, while the present invention has been described with reference to various illustrative examples thereof, it is not intended that the present invention be limited to these specific examples. Those skilled in the art will recognize that variations, modifications, and combinations of the disclosed subject matter can be made, without departing from the spirit and scope of the present invention, as set forth in the appended claims.

What is claimed is:

1. A mobile point-of-care medical station, comprising:
a patient medical parameter processor for automatically acquiring data representing medical parameters of a patient and processing said patient medical parameter data for presentation to a user on a display; and
a communication interface, enabling communication with remote systems via a network, for communicating patient medical parameter data to a remote system by formatting patient medical parameter data into a plurality of individual messages and an individual message incorporates,
a patient identifier and
a communication address associated with a particular communication interface of a particular mobile point-of-care medical station enabling said remote system to identify said particular mobile point-of-care medical station from a plurality of different mobile point-of-care medical stations.

2. A system according to claim 1, wherein
said communication interface enables communication with remote systems via a wireless network.

3. A system according to claim 1, wherein
said communication interface formats patient medical parameter data of an individual message to incorporate a port address associated with said particular communication interface of said particular mobile point-of-care medical station.

4. A system according to claim 1, wherein
said remote system comprises an executable application executing on a server.

5. A system according to claim 1, wherein
said communication interface formats patient medical parameter data into a plurality of individual messages compatible with an Health Level 7 (HL7) transaction data format.

6. A system for communicating with a plurality of mobile point-of-care medical stations, comprising:
a repository associating a plurality of mobile point-of-care medical station identifiers with a corresponding plurality of communication interface communication addresses;
a first communication interface, enabling communication with a plurality of mobile point-of-care medical stations via a network, for receiving patient medical parameter data from an individual medical station formatted as an individual message incorporating a patient identifier and a communication address associated with a particular communication interface of a particular mobile point-of-care medical station; and
a message processor using said repository for determining a particular mobile point-of-care medical station associated with a particular received individual message.

7. A system according to claim 6, including
a second communication interface enabling communication of received data to a medical information system.

8. A system according to claim 7, wherein
said first and second communication interfaces employ first and second separate operational processes respectively,
said first process managing inbound communications from mobile point-of-care medical stations and
said second process managing outbound communications to said medical information system, said separate first and second processes preventing data queuing interaction between inbound and outbound communications.

9. A system according to claim 8, wherein
said separate first and second processes are separate threads of operation.

10. A system according to claim 6, wherein
said first communication interface receives patient medical parameter data and associated local workstation identification information to uniquely establish a link between patient, medical monitor, and raw parameters ensuring no loss of patient specificity.

11. A system according to claim 6, wherein
said formatted individual message is compatible with an Health Level 7 (HL7) transaction data format.

12. A method for operating mobile point-of-care medical station, comprising the steps of:
automatically acquiring data, representing medical parameters of a patient, and processing said patient medical parameter data for presentation to a user on a display; and
enabling communication with remote systems via a network, for communicating patient medical parameter data to a remote system by formatting patient medical parameter data into a plurality of individual messages, wherein an individual message incorporates,
a patient identifier and
a communication address associated with a particular communication interface of a particular mobile point-of-care medical station enabling said remote system to identify said particular mobile point-of-care medical station from a plurality of different mobile point-of-care medical stations.

13. A method according to claim 12, wherein
said step of enabling formats patient medical parameter data of an individual message to incorporate a port address associated with said particular communication interface of said particular mobile point-of-care medical station.

14. A method according to claim 12, wherein
said communication interface formats patient medical parameter data into a plurality of individual messages compatible with an Health Level 7 (HL7) transaction data format.

15. A method for communicating with a plurality of mobile point-of-care medical stations, comprising the steps of:
storing data associating a plurality of mobile point-of-care medical station identifiers with a corresponding plurality of communication interface communication addresses;
enabling a first communication with a plurality of mobile point-of-care medical stations via a network, and receiving patient medical parameter data from an individual medical station formatted as an individual message incorporating a patient identifier and a communication address associated with a particular communication interface of a particular mobile point-of-care medical station; and
using said repository for determining a particular mobile point-of-care medical station associated with a particular received individual message.

16. A method according to claim 15, further comprising the step of:
enabling a second communication of received data to a medical information system.

17. A method according to claim 16, wherein
said steps of enabling a first and second communication employ first and second separate operational processes, respectively,
said first process managing inbound communications from mobile point-of-care medical stations, and
said second process managing outbound communications to said medical information system, said separate first and second processes preventing data queuing interaction between inbound and outbound communications.

18. A method according to claim 17, wherein
said separate first and second processes are separate threads of operation.

19. A method according to claim 15, wherein
said step of enabling a first communication further comprises receiving patient medical parameter data and associated local workstation identification information to uniquely establish a link between patient, medical monitor, and raw parameters ensuring no loss of patient specificity.

20. A method according to claim 15, wherein
said formatted individual message is compatible with an Health Level 7 (HL7) transaction data format.
